(19) United States
(12) Patent Application Publication
(10) Pub. No.: US 2026/0069278 A1
(43) Pub. Date: Mar. 12, 2026

(54) PROTECTING CORONARY ARTERIES AND MYOCARDIUM BLOOD VESSELS DURING MEDICAL PROCEDURES

(71) Applicant: Zeev BRANDEIS, Rosh HaAyin (IL)

(72) Inventor: Zeev BRANDEIS, Rosh HaAyin (IL)

(21) Appl. No.: 19/106,314

(22) PCT Filed: Aug. 24, 2023
(86) PCT No.: PCT/IL2023/050901

§ 371 Date: Feb. 25, 2025

Related U.S. Application Data
(63) Continuation-in-part of application No. PCT/IL2023/050875, filed on Aug. 17, 2023.
(60) Provisional application No. 63/398,546, filed on Aug. 17, 2022.
(60) Provisional application No. 63/400,771, filed on Aug. 25, 2022.
(60) Provisional application No. 63/400,773, filed on Aug. 25, 2022.
(60) Provisional application No. 63/400,779, filed on Aug. 25, 2022.
(60) Provisional application No. 63/400,866, filed on Aug. 25, 2022.

(30) Foreign Application Priority Data
Aug. 17, 2023 (WO) PCT/IL2023/050875

(51) Int. Cl. A61B 17/12; A61B 17/00; A61B 90/00

(52) U.S. Cl. CPC A61B 17/12136; A61B 17/12168; A61B 2017/00871; A61B 2090/3966

(57) ABSTRACT

A device for protecting debris from entering coronary arteries, the device including an expandable frame, and a debris blocking obstacle, wherein the expandable frame is shaped and sized to expand against walls of an ascending aorta. A method of reducing damage to the heart during a vascular procedure including identifying a potentially debris-causing event, and reducing debris carried by backflow from an aorta into a coronary artery during diastole in response to the identifying. A method for protecting debris from entering coronary arteries during a medical procedure, the method including inserting a device for protecting debris from entering coronary arteries into an aorta of a patient, positioning the device to protect debris from entering the coronary arteries, and performing the medical procedure on a heart of the patient. Related apparatus and methods are also described.

### RELATED APPLICATIONS

This claims the benefit of priority of U.S. Provisional Patent Application No. 63/400,771 filed Aug. 25, 2022, titled “PROTECTING CORONARY ARTERIES AND MYOCARDIUM BLOOD VESSELS DURING MEDICAL PROCEDURES”; of U.S. Provisional Patent Application 63/400,773 filed Aug. 25, 2022, titled “MANAGING DEBRIS-PROTECTION IN RELATION TO VASCULAR MEDICAL PROCEDURES OR MEDICAL PROCEDURES TO A HEART” and of U.S. Provisional Patent Application 63/400,779 filed Aug. 25, 2022, titled “FIXATION OF DEBRIS PROTECTION DEVICES IN BODY LUMENS”.

This application is also a Continuation-in-Part (CIP) of PCT Patent Application No. PCT/IL2023/050875 having International filing date of Aug. 17, 2023, which claims the benefit of priority of U.S. Provisional Patent Application No. 63/398,546 filed on Aug. 17, 2022, titled “CAPTURING FLOWING DEBRIS IN BLOOD VESSELS AND OTHER BODY LUMENS”.

The contents of all of the above applications are incorporated by reference as if fully set forth herein.

### FIELD AND BACKGROUND

The present disclosure, in some embodiments thereof, relates to devices and methods to protect coronary arteries from entry of debris and, more particularly, but not exclusively, to devices and methods to protect coronary arteries from entry of debris during medical procedures.

Blood borne debris such as emboli, calcification particles, plaque and detached plaque particles, tissue particles like myocardial tissue endothelial tissue and others, particles from foreign sources-metals, polymers and others can be dangerous, the debris may cause a pathological event, for example accompanying illness or injury or even medical procedures.

For example, in trans-catheter aortic valve implantation (TAVI), particles were found in the brain in 99% of patients by diffusion-weighted magnetic resonance imaging (DWI or DW-MRI). There was a significantly higher amount of debris related to the vascular bed (valve tissue, arterial wall, calcification), and additional particles: atherosclerotic plaque, myocardial tissue, endothelium tissue, and foreign particles of unknown source-such as polymers and gas and/or air bubbles. A source of these particles source is probably the left ventricle and the area where TAVI was performed. Special attention is given to micro-emboli, namely small particles in a size range of 150 micron to 120 micron down to a range of 50 micron to 30 micron. The micro-emboli particles are suspected to be prone to migrate to the arterioles and to become embedded there.

Additional background art includes:

International Patent Application Publication Number WO 2019/064223 of Brandeis, which describes an aortic protection device including a mesh lumen shaped and sized to extend along the aorta, from a heart-side of a brachiocephalic artery exit from the aorta to distal of a left subclavian artery exit from the aorta, wherein the mesh lumen is arranged to change a porosity of mesh pores in response to external control.

The disclosures of all references mentioned above and throughout the present specification, as well as the disclosures of all references mentioned in those references, are hereby incorporated herein by reference.

### SUMMARY

The present disclosure, in some embodiments thereof, relates to devices and methods to protect coronary arteries and cardiac vasculature, for example cardiac arteries, from entry of debris and, more particularly, but not exclusively, to devices and methods to protect coronary arteries and cardiac vasculature from entry of debris during medical procedures.

According to an aspect of some embodiments of the present disclosure there is provided a device for protecting debris from entering coronary arteries, the device including an expandable frame, and a debris blocking obstacle, wherein the expandable frame is shaped and sized to expand against walls of an ascending aorta.

According to some embodiments of the disclosure, the debris blocking obstacle includes a solid section of the frame at the entries to the coronary arteries, enabling to block blood flow to the coronary arteries when the frame is expanded.

According to some embodiments of the disclosure, the debris blocking obstacle includes an expandable balloon having a hollow tubular shape, the balloon shaped and sized to such that when the balloon is expanded, the balloon presses against aorta walls and blocks blood flow to the coronary arteries.

According to some embodiments of the disclosure, the balloon includes flexible protrusions shaped and sized so that when the balloon is not inflated, the protrusions maintain a flow path for blood to flow around the balloon.

According to some embodiments of the disclosure, the debris blocking obstacle includes a mesh with pores sized to prevent particles larger than 30 microns+/−10 microns to pass therethrough, the mesh attached to the frame, wherein when the frame is expanded against walls of the ascending aorta at exits to coronary arteries, the mesh filters blood entering coronary arteries.

According to some embodiments of the disclosure, the mesh is designed such that a pore size of the mesh can be controlled to change.

According to some embodiments of the disclosure, the device includes a wire for controlling the pore size of the mesh, the wire sized to extend outside of a patient's body.

According to some embodiments of the disclosure, the mesh is designed to change pore size from a range of 120-150 microns to a range of 30-50 microns.

According to some embodiments of the disclosure, the mesh is designed to completely close.

According to some embodiments of the disclosure, the debris blocking obstacle includes a one way obstacle shaped and sized to prevent particles from being swept back upstream toward coronary arteries of a heart during diastole.

According to some embodiments of the disclosure, the one way obstacle includes a one way valve.

According to some embodiments of the disclosure, the one way obstacle includes a flaps enabling flow downstream and blocking flow upstream.

According to some embodiments of the disclosure, the one way obstacle includes a mesh which prevent upstream flow of debris.

According to some embodiments of the disclosure, including a control wire for controlling activation and de-activation of the one way obstacle.

According to some embodiments of the disclosure, the one way obstacle includes flaps which, when blood attempts to flow back upstream during diastole, the flaps are swept by the blood and extend across the aorta, blocking backward flow of the blood.

According to some embodiments of the disclosure, the one way obstacle includes mesh flaps which, when blood attempts to flow back upstream during diastole, the flaps are swept by the blood and extend across the aorta, blocking backward flow of debris and allowing at least some of the blood to flow back.

According to some embodiments of the disclosure, the one way obstacle includes both impermeable flaps and mesh flaps, which, when blood attempts to flow back upstream during diastole, the flaps are swept by the blood and extend across the aorta, the impermeable flaps obstructing backward flow of blood, and the mesh flaps allowing at least some blood to flow back.

According to some embodiments of the disclosure, the impermeable flaps and the mesh flaps are individually controllable.

According to some embodiments of the disclosure, the obstacle is designed such that the obstacle can be activated to change from obstructing particles from being swept back upstream during diastole to not obstructing particles from being swept back upstream during systole.

According to some embodiments of the disclosure, the obstacle includes radio opaque markers to enable detecting whether the obstacle is activated to block or not.

According to some embodiments of the disclosure, the device includes a combination of more than one type of debris blocking obstacle.

According to some embodiments of the disclosure, the device is configured to be integrated with a TAVI/TAVR delivery system.

According to some embodiments of the disclosure, the device is configured to slide along a TAVI/TAVR guide wire.

According to some embodiments of the disclosure, the device is configured to be integrated with a cerebral embolic protection (CEP) or embolic protection device (EPD).

According to some embodiments of the disclosure, the frame includes shape memory metal.

According to some embodiments of the disclosure, the frame includes polymer.

According to some embodiments of the disclosure, the device is configured to enable medical tools to pass along the device and upstream of the device when the device is located in an aorta.

According to some embodiments of the disclosure, the device is configured to enter a patient's body together with medical tools for operating on a heart.

According to some embodiments of the disclosure, the frame is configured such that expansion of the frame against walls of the aorta anchors the device to resist movement along a direction of blood flow.

According to an aspect of some embodiments of the present disclosure there is provided a method of reducing damage to the heart during a vascular procedure including identifying a potentially debris-causing event, and reducing debris carried by backflow from an aorta into a coronary artery during diastole in response to the identifying.

According to some embodiments of the disclosure, the identifying includes identifying a step of positioning a prosthesis performed in a prosthesis implant procedure.

According to some embodiments of the disclosure, the identifying includes identifying a step of re-positioning a prosthesis performed in a prosthesis implant procedure.

According to some embodiments of the disclosure, the identifying includes identifying a step of pace-up performed in a prosthesis implant procedure.

According to some embodiments of the disclosure, the identifying includes identifying a step of pace-down performed in a prosthesis implant procedure.

According to some embodiments of the disclosure, the identifying includes identifying a step of inflating a balloon performed in a prosthesis implant procedure.

According to some embodiments of the disclosure, the reducing debris includes blocking blood entrance to coronary arteries.

According to some embodiments of the disclosure, the reducing debris includes filtering blood entering coronary arteries.

According to some embodiments of the disclosure, the reducing debris includes activating a one-way valve preventing blood from flowing back from the aorta to the coronary artery.

According to some embodiments of the disclosure, the reducing debris includes activating a one-way filter preventing blood from flowing back from the aorta to the coronary artery.

According to an aspect of some embodiments of the present disclosure there is provided a method for protecting debris from entering coronary arteries during a medical procedure, the method including inserting a device for protecting debris from entering coronary arteries into an aorta of a patient, positioning the device to protect debris from entering the coronary arteries, and performing the medical procedure on a heart of the patient.

According to some embodiments of the disclosure, the inserting includes inserting by femoral access.

According to some embodiments of the disclosure, the inserting includes inserting by radial access.

According to some embodiments of the disclosure, the inserting includes inserting by surgical access.

According to some embodiments of the disclosure, the inserting includes inserting by hybrid access, a combination of surgical access and catheter insertion.

According to some embodiments of the disclosure, further including extracting the device from the body.

According to some embodiments of the disclosure, further including activating the device to protect debris from entering the coronary arteries during the medical procedure.

According to some embodiments of the disclosure, the activating to protect debris from entering coronary arteries is performed together with performing a pace-up on the patient's heart.

According to some embodiments of the disclosure, the activating to protect debris from entering coronary arteries is performed together with performing a pace-down on the patient's heart.

According to some embodiments of the disclosure, the device is inserted together with tools for performing the cardiac procedure.

According to some embodiments of the disclosure, the device is inserted before tools for performing the cardiac procedure.

According to some embodiments of the disclosure, the device is inserted after tools for performing the cardiac procedure.

According to some embodiments of the disclosure, the device includes a frame sized and shaped to have solid walls at exits to coronary arteries, and the activating include controlling the frame to expand against walls of an ascending aorta so that the solid walls of the frame block blood flow to exits to coronary arteries.

According to some embodiments of the disclosure, the device includes a frame sized and shaped to have an arrangement of pores at exits to coronary arteries, and the activating include controlling the frame to expand against walls of an ascending aorta so that the pores of the frame block particle flow to exits to coronary arteries.

Unless otherwise defined, all technical and/or scientific terms used herein have the same meaning as commonly understood by one of ordinary skill in the art to which the disclosure pertains. Although methods and materials similar or equivalent to those described herein can be used in the practice or testing of embodiments of the disclosure, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the disclosure are herein described, by way of example only, with reference to the accompanying drawings. With specific reference now to the drawings in detail, it is stressed that the particulars shown are by way of example and for purposes of illustrative discussion of embodiments of the disclosure. In this regard, the description taken with the drawings makes apparent to those skilled in the art how embodiments of the disclosure may be practiced.

In the drawings:

FIGS. 1A and 1B are simplified line drawing illustrations of a human aorta;

FIG. 2A is a simplified line drawing illustration of a device for preventing back flow of blood according to an example embodiment;

FIG. 2B is a simplified line drawing illustration of a device for preventing back flow of blood according to an example embodiment;

FIG. 2C is a simplified line drawing illustration of a device for preventing blood from entering coronary arteries according to an example embodiment;

FIG. 2D is a simplified line drawing illustration of a device for preventing blood from entering coronary arteries according to an example embodiment;

FIG. 2E is a simplified line drawing illustration of a device for preventing blood from entering coronary arteries according to an example embodiment;

FIG. 2F is a simplified line drawing illustration of a device for preventing back flow of debris according to an example embodiment;

FIG. 3A is a simplified line drawing illustration of a coronary artery protection device located in an aorta in conjunction with a debris-protection device for preventing debris from entering brain arteries according to an example embodiment;

FIG. 3B is a simplified line drawing illustration of a coronary artery protection device located in an aorta in conjunction with a debris capture device, according to an example embodiment;

FIG. 3C is a simplified line drawing illustration of a coronary artery protection device located in an aorta in conjunction with a debris capture device and an aortic protection device, according to an example embodiment;

FIG. 3D is a simplified line drawing illustration of a coronary artery protection device located in an aorta in conjunction with a debris capture device and an aortic protection device, according to an example embodiment;

FIG. 4A is a simplified flow chart illustration of a method of reducing damage to the heart during a vascular procedure, according to an example embodiment;

FIG. 4B is a simplified flow chart illustration of a method for protecting debris from entering coronary arteries during a medical procedure according to an example embodiment;

FIG. 5A is a simplified flow chart illustration of a method for preventing back flow of blood into coronary arteries during a medical procedure according to an example embodiment;

FIG. 5B is a simplified flow chart illustration of a method for protecting coronary arteries from entry of particles during a medical procedure according to an example embodiment;

FIG. 6A is a simplified line drawing illustration of a device protecting coronary arteries according to an example embodiment;

FIG. 6B is a simplified line drawing illustration of a device protecting coronary arteries according to an example embodiment;

FIG. 6C is a simplified line drawing illustration of devices protecting coronary arteries according to an example embodiment;

FIG. 6D is a simplified line drawing illustration of devices protecting coronary arteries according to an example embodiment;

FIG. 6E is a simplified line drawing illustration of a device for protecting coronary arteries according to an example embodiment;

FIG. 6F is a simplified line drawing illustration of a device for protecting coronary arteries according to an example embodiment; and

FIG. 6G is a simplified line drawing illustration of a device for protecting coronary arteries according to an example embodiment.

### DESCRIPTION OF SPECIFIC EMBODIMENTS

The present disclosure, in some embodiments thereof, relates to devices and methods to protect coronary arteries from entry of debris and, more particularly, but not exclusively, to devices and methods to protect coronary arteries from entry of debris during medical procedures.

### Introduction

Micro emboli, including small particles, may enter the coronary arteries and reach small arterial vessels including the arterioles. Such small particles have a role in a cascade of events in the arterioles, leading to myocardial fibrosis. Myocardial fibrosis is found in post-mortem studies of patients' hearts who had TAVI before their death, and may have been the reason for sudden death in patients that underwent the TAVI procedure. Micro emboli and other particle types are also part of a myocardial cascade which leads to myocardium tissue inflammation and necrosis in myocardium tissue. A connection was found between atrial fibrillation and cardiac arrhythmia.

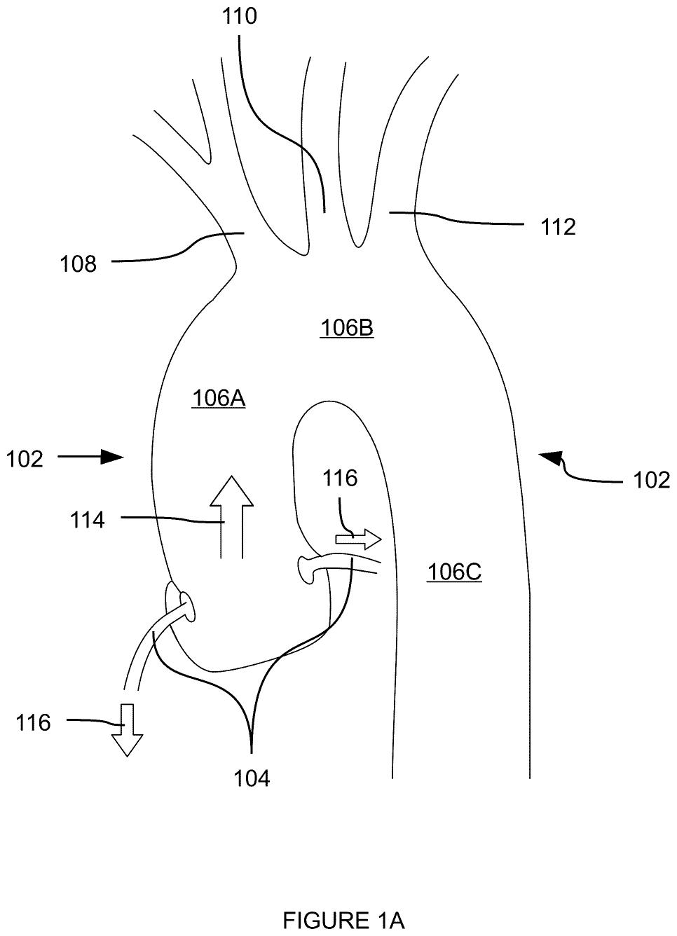

For purposes of better understanding some embodiments of the present disclosure, reference is first made to the operation of typical blood flow as illustrated in FIGS. 1A and 1B.

FIGS. 1A and 1B are simplified line drawing illustrations of a human aorta.

FIG. 1A is intended to show the aorta during diastole and FIG. 1B is intended to show the aorta during systole.

First, details regarding blood vessels are provided. FIGS. 1A and 1B show the aorta 102, and the following arteries branching off from the aorta: coronary arteries 104, the brachiocephalic artery 108 (commonly referred to clinically as the innominate artery), the left common carotid artery 110 and the left subclavian artery 112. Portions of the aorta 102 are named an ascending aorta 106A, aortic arch 106B and descending aorta 106C.

FIG. 1A shows blood flowing 114 downstream along the aorta during diastole, and blood flowing 116 into the coronary arteries 104.

FIG. 1B shows blood flowing 118 downstream and also flowing back upstream during systole, and blood flowing 120 into the coronary arteries 104.

The aorta walls are relatively flexible. During diastole blood at relatively high pressure is pumped from the heart and causes an expansion of the aorta. During systole, the aorta compresses, further pumping blood downstream, but also providing some backflow of the blood, as indicated by the double ended arrow 118 in FIG. 1B.

### Overview

The backflow may cause some debris which sped past the entrances to the coronary arteries 104 to flow back and potentially enter the coronary arteries.

It is desirable to prevent the backflow debris from entering the coronary arteries.

It is noted that the amount of blood reaching the coronary arterial vasculature during diastole is significantly higher by volume, relative to during systole, because during systole the arteries are compressed and their volume is reduced.

It is also desirable in general to prevent blood-borne debris from entering the coronary arteries, both during diastole (backflow) and during systole.

The Windkessel effect is a term used in medicine to account for the shape of the arterial blood pressure waveform in terms of interaction between stroke volume and the compliance of the aorta and large elastic arteries (Windkessel vessels) and the resistance of smaller arteries and arterioles.

It is noted that during medical procedures, and especially during medical procedures to the heart, debris may be released or produced. Some of the debris may flow into the coronary arteries, and pose a potential risk. It is desirable to prevent debris from flowing into the coronary arteries.

The term debris is used throughout the present specification and claims to include blood clots, emboli, micro-emboli, calcification particles, body tissue particles, myocardial tissue particles, polymer particles, polymer micro-particles, foreign particles in body lumens, endothelium tissue particles, unorganized blood clots, organized blood clots, plaque particles, particles flowing in the blood stream and other undesirable particles found in body fluids. The term debris also includes stones from various body sources-kidney stones, gallbladder stones, spleen, pancreatic stones and others.

### Coronary Protection Devices Blocking Backflow of Debris

An aspect of some embodiments relates to a device for blocking backflow of debris from the aorta toward the coronary arteries.

Preventing backflow may reduce the amount of blood reaching the heart via the coronary arteries. However, the amount of blood reaching the coronary arterial vasculature during diastole is significantly higher by volume, relative to during systole, because during systole the arteries are compressed and their volume is reduced. The heart can withstand such a reduction in blood flow to the coronary arterial vasculature for a short period of time. By way of a non-limiting example the duration of the short period of time can be equal to the duration of a Rapid Ventricular Pacing (RVP) step in a TAVI procedure, also called a “Pace up” step, where a patient's pulse is raised to a point of tachycardia—which provides little or no blood supply to the heart and brain.

Preventing backflow may reduce almost completely the amount of blood reaching the heart via the coronary arteries. In some embodiments, activation of backflow prevention is optionally performed at a specific period of time, as will be described below.

A non-limiting example embodiment of such a device is a device, such as, by way of a non-limiting example, an expandable frame shaped and sized to place a one way obstacle shaped and sized to prevent particles from being swept back upstream toward coronary arteries of a heart, during diastole.

A non-limiting example embodiment of such a device is a device, such as, by way of a non-limiting example, an expandable frame shaped and sized to place a one way valve shaped and sized to prevent particles from being swept back upstream toward coronary arteries of a heart, during diastole.

In some embodiments, the one way valve optionally includes flexible leaflets which prevent backflow of blood.

In some embodiments, the flexible leaflets are non-permeable leaflets.

In some embodiments, the flexible leaflets are mesh leaflets, with a pore size configured to prevent a certain size of debris from flowing back, while potentially enabling blood without such debris to flow back.

In some embodiments, the one way obstacle or the one way valve is optionally configured to be controlled to activate one way blocking or filtering and to de-activate the blocking/filtering.

In some embodiments, the device for preventing backflow optionally includes debris traps. In some embodiments, the debris traps may optionally be opened and/or closed by control from outside a patient's body. In some embodiments, the debris traps are optionally closed before extracting from the patient's body, potentially extracting the trapped debris from the patient's body.

In some embodiments, the device for blocking backflow of debris is shaped and sized for delivery through a catheter sized in a range of 5-8 French.

### Coronary Protection Devices Blocking Debris Entry to Coronary Arteries

An aspect of some embodiments relates to a device for blocking debris from entering the coronary arteries.

A non-limiting example embodiment of such a device is an expandable frame shaped and sized to expand against walls of the ascending aorta at entrances to the coronary arteries, wherein the frame comprises a solid wall at the entrances to the coronary arteries, enabling to block blood flow to the coronary arteries when the frame is expanded. The blocking is optionally performed at a specific period of time, as will be described below.

A non-limiting example embodiment of such a device is an expandable balloon shaped and sized to expand against walls of the ascending aorta at entrances to the coronary arteries, wherein the balloon blocks the entrances to the coronary arteries, enabling to block blood flow to the coronary arteries when the balloon is expanded. Expanding the balloon is optionally performed at a specific period of time, as will be described below.

A non-limiting example embodiment of such a device is a device, such as, by way of a non-limiting example, an expandable frame shaped and sized to place a mesh at entrances to the coronary arteries, wherein the mesh can prevent debris above a certain size, for example the pore size of the mesh, enabling to block such debris from flowing into the coronary arteries when the mesh is activated. In some embodiments, the activation is optionally performed at a specific period of time, as will be described below.

It is noted that the filtering at entrances to the coronary arteries is especially relevant during diastole, which is when a greater volume of blood typically enters the coronary arteries, as also noted above

In some embodiments, the device for blocking debris optionally includes debris traps. In some embodiments, the debris traps may optionally be opened and/or closed by control from outside a patient's body. In some embodiments, the debris traps are optionally closed before extracting from the patient's body, potentially extracting the trapped debris from the patient's body.

In some embodiments, the device for blocking debris from entering the coronary arteries is shaped and sized for delivery through a catheter sized in a range of 5-8 French.

### Timing of the Blocking

An aspect of some embodiments relates to when a device is activated to block debris from entering the coronary arteries and/or for blocking backflow of blood toward the coronary arteries.

In some embodiments, the activation is performed when debris is expected to be produced during a medical procedure, for example a medical procedure on the heart.

In some embodiments, the activation is performed when debris is expected to be released during a medical procedure, for example a medical procedure on the heart.

In some embodiments, the activation is performed during a specific step of a medical procedure.

By way of a non-limiting example, when TAVI is performed, there is a step called “pace-up”, or Rapid Ventricular Pacing (RVP). Rapid ventricular pacing (RVP) is a step during TAVR or TAVI which is used for temporary reduction in cardiac output during the procedure.

During RVP a replacement heart valve is deployed. The deployment can produce and/or release debris.

In some embodiments, the blocking is performed approximately (simultaneously with or a short time after) pace-up. During such a time the heart is not producing much output, and what output there is may contain debris, and it is potentially beneficial to activate blocking debris from entering the coronary arteries and/or even to activate blocking backflow of blood toward the coronary arteries.

By way of a non-limiting example, when TAVI is performed, there is a step called “pace-down”, or “stop RVP”, when the heart is paced back down from “pace-up”, and cardiac output goes back up. During such a step there is a danger of a potential “debris storm”, a large amount of debris being produced and/or released and flowing downstream. In some embodiments, and it is potentially beneficial to activate blocking debris from entering the coronary arteries and/or even to activate blocking backflow of blood toward the coronary arteries.

In some embodiments, the blocking is performed approximately (simultaneously with or even a short time before) pace-down.

In some embodiments, the activation is optionally performed by a physician as part of a medical procedure, to protect the coronary arteries from debris.

In some embodiments, the activation is optionally performed automatically based on sensor input(s).

By way of a non-limiting example, a heart rate sensor may optionally detect pace-up and automatically activate blocking debris from entering the coronary arteries and/or to activate blocking backflow of blood toward the coronary arteries.

By way of a non-limiting example, a heart rate sensor may optionally detect pace-down and automatically activate blocking debris from entering the coronary arteries and/or to activate blocking backflow of blood toward the coronary arteries.

In some embodiments, the activation of blocking debris from entering the coronary arteries and/or blocking backflow of blood toward the coronary arteries, may be limited in time.

In some embodiments, a physician limits the activation to no more than 10 seconds, 20 seconds, 30 seconds, 60 seconds, 90 seconds or 120 seconds.

In some embodiments, an automatic timer limits the activation to no more than 30 seconds, 60 seconds, 90 seconds or 120 seconds.

Coronary Protection Devices Operating in Conjunction with Additional Debris-Protection Devices

An aspect of some embodiments relates to operating a device for blocking debris from entering the coronary arteries and/or for blocking backflow of blood toward the coronary arteries in conjunction with additional debris-protection devices.

In some embodiments, the coronary protection device may optionally be used in conjunction with a debris capture device, by way of a non-limiting example such as described in above-mentioned U.S. Provisional Patent Application No. 63/398,546 by Brandeis.

In some embodiments, the coronary protection device may optionally be used in conjunction with an aortic protection device for protecting brain arteries, by way of a non-limiting example such as described in above-mentioned International Patent Application Publication Number WO 2019/064223 of Brandeis.

Coronary Protection Devices Operating in Conjunction with Additional Medical Devices

An aspect of some embodiments relates to operating a device for blocking debris from entering the coronary arteries and/or for blocking backflow of blood toward the coronary arteries in conjunction with additional medical devices

In some embodiments, the coronary protection device extends, in use, to walls of the aorta, leaving a center space or lumen for other medical tools to pass therethrough.

In some embodiments, the coronary protection device may optionally be configured to be part of medical tools, such as, by way of a non-limiting example, TAVI tools, and slide along the TAVI tool to reach the location of the coronary arteries.

In some embodiments, the coronary protection device is optionally inserted via an existing delivery system, by way of a non-limiting example a delivery system for TAVI/TAVR.

In some embodiments, the coronary protection device is configured to slide along an existing TAVI/TAVR delivery system.

In some embodiments, the coronary protection device may optionally be configured to be part of medical tools, such as, by way of a non-limiting example, TAVI tools, and fixed to a portion of the TAVI tool to reach the location of the coronary arteries.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

Before explaining at least one embodiment of the disclosure in detail, it is to be understood that the disclosure is not necessarily limited in its application to the details set forth in the following description or exemplified by the examples. The disclosure is capable of other embodiments or of being practiced or carried out in various ways.

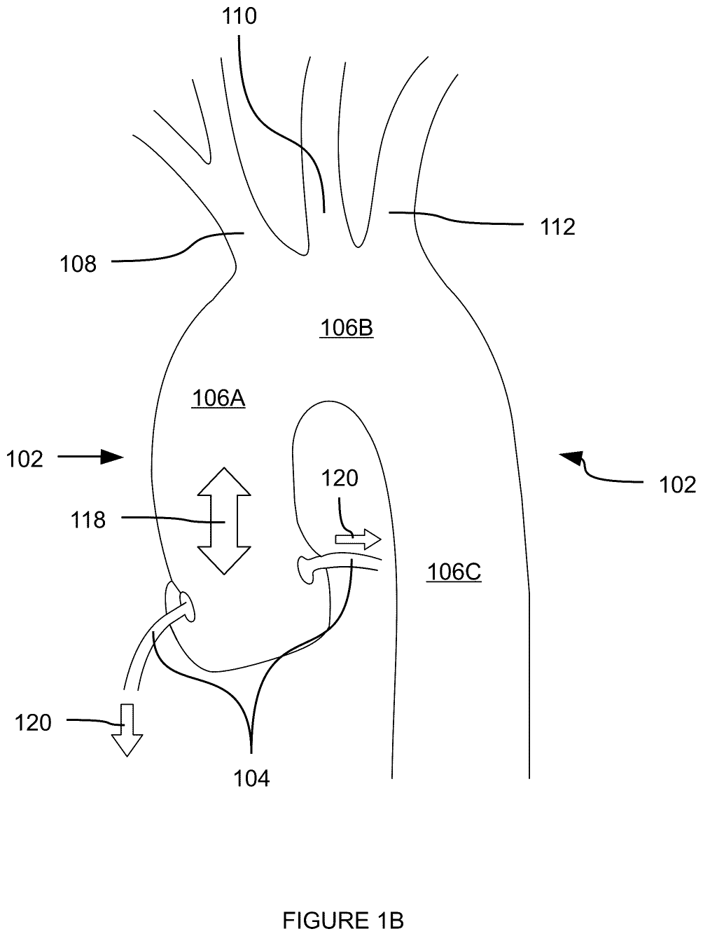

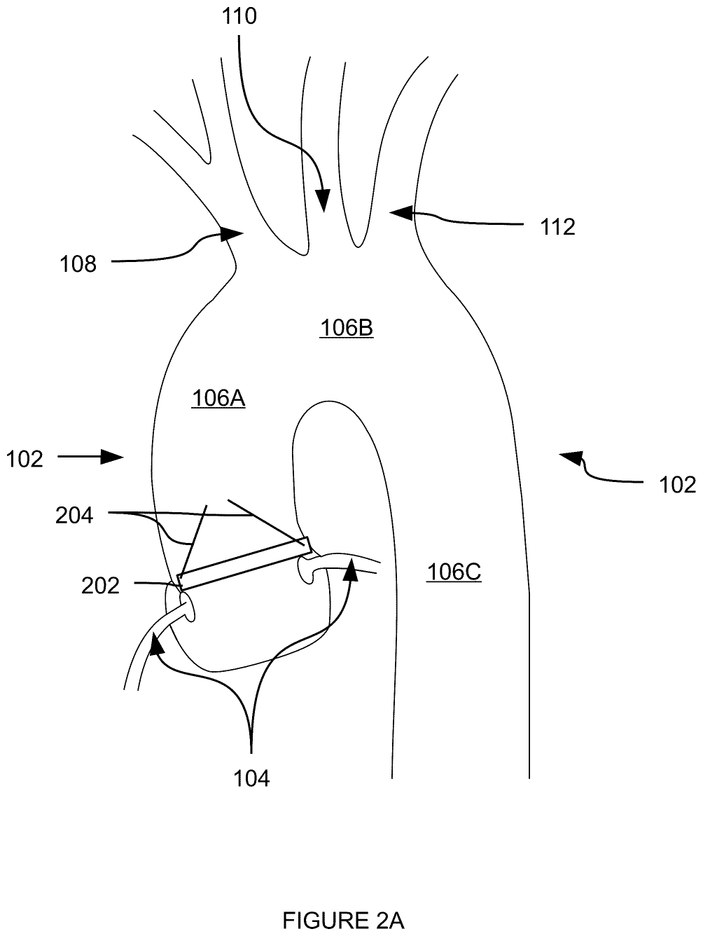

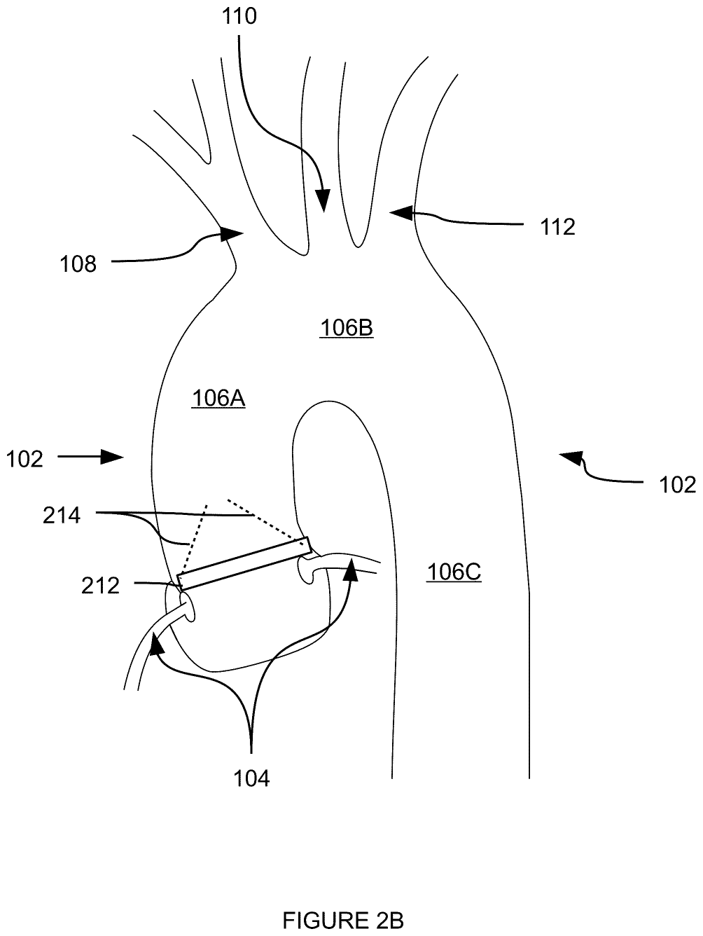

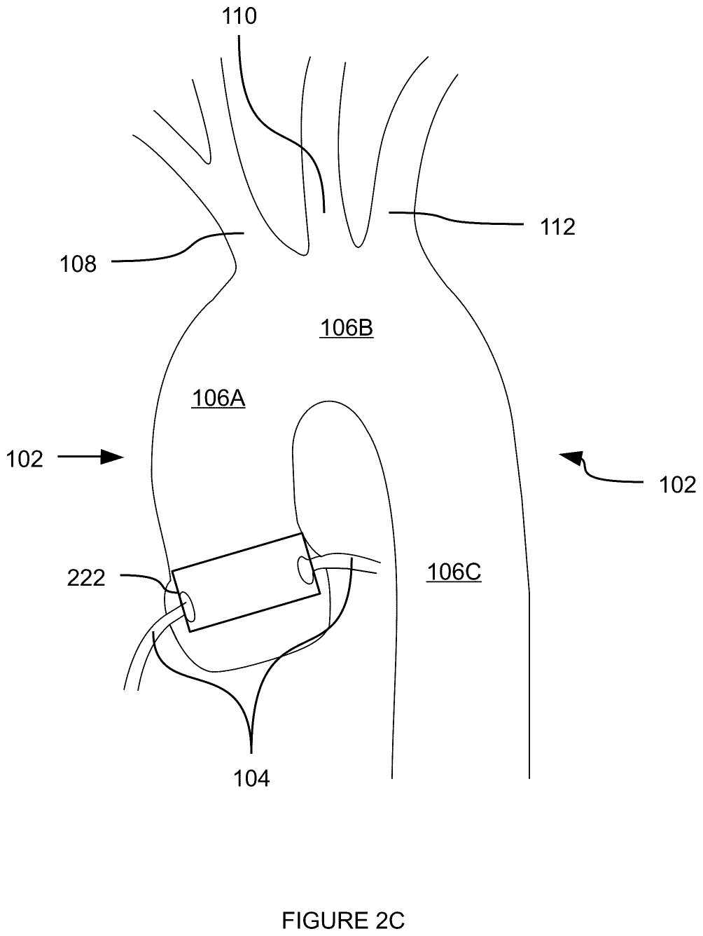

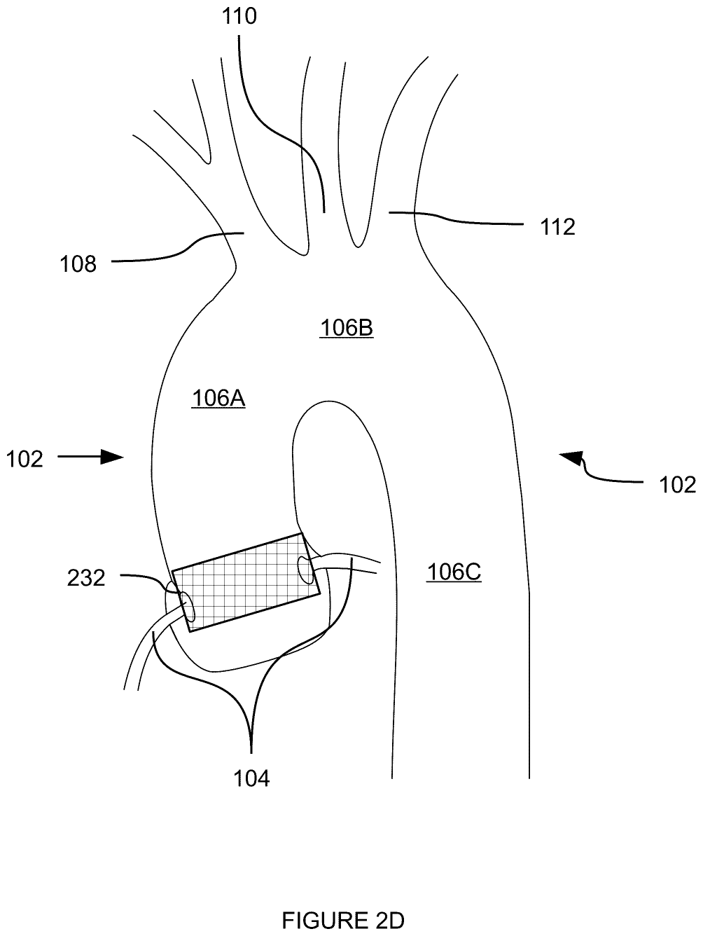

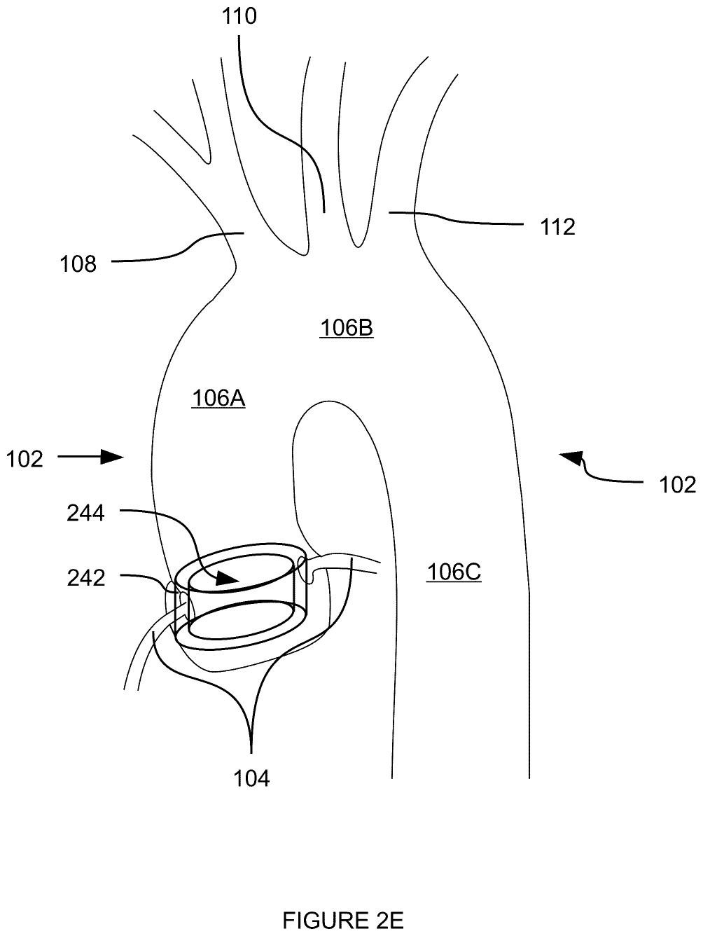

Reference is now made to FIG. 2A, which is a simplified line drawing illustration of a device for preventing back flow of blood according to an example embodiment.

FIG. 2A is intended to show a device which operates as a one way flow valve.

FIG. 2A shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 2A shows a device 202 for preventing back flow of blood located just downstream of the coronary arteries 104.

FIG. 2A shows the device 202 including a one way valve, for example including leaflets 204 configured to prevent back flow of blood.

In some embodiments, the leaflets 204 are flexible, enabling passage of medical tool(s) (not shown) between the leaflets 204, and yet, when the leaflets are activated to block back flow, the leaflets 204 can lie against the medical tool(s), blocking a space between the leaflets 204 and the medical tool(s), and block blood from flowing back.

Reference is now made to FIG. 2B, which is a simplified line drawing illustration of a device for preventing back flow of blood according to an example embodiment.

FIG. 2B is intended to show a device which operates as a one way filtering valve.

FIG. 2B shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 2B shows a device 212 for preventing back flow of blood located just downstream of the coronary arteries 104.

FIG. 2B shows the device 212 including a one way valve, for example including leaflets 214 configured to prevent back flow of blood.

In some embodiments, the leaflets 214 are flexible and include a mesh. The mesh filters back flow blood, preventing debris particles from flowing back, and allowing blood to flow back.

In some embodiments, the size of pores in the mesh is selected to be in a range of 120 to 30 microns.

Reference is now made to FIG. 2C, which is a simplified line drawing illustration of a device for preventing blood from entering coronary arteries according to an example embodiment.

FIG. 2C is intended to show a device which blocks entrance of blood to the coronary arteries.

FIG. 2C shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 2C shows a device 222 for preventing blood from entering the coronary arteries 104.

FIG. 2C shows the device 222, for example an expandable frame 222 shaped and sized to expand against walls of an ascending aorta at exits to coronary arteries, the frame including a solid wall at least at entries to the coronary arteries 104, enabling to block blood flow to the coronary arteries when the frame 222 is expanded and located such that the solid wall is at the entrance to the coronary arteries 104.

Reference is now made to FIG. 2D, which is a simplified line drawing illustration of a device for preventing blood from entering coronary arteries according to an example embodiment.

FIG. 2D is intended to show a device which filters blood entering the coronary arteries, blocking debris from entering the coronary arteries 104.

FIG. 2D shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 2D shows a device 232 for filtering blood entering the coronary arteries 104.

FIG. 2D shows the device 232, for example an expandable frame 232 shaped and sized to expand against walls of an ascending aorta at exits to coronary arteries, the frame including a mesh filter at least at entries to the coronary arteries 104, enabling to filter blood flow to the coronary arteries when the frame 232 is expanded and located such that the filter is at the entrance to the coronary arteries 104.

Reference is now made to FIG. 2E, which is a simplified line drawing illustration of a device for preventing blood from entering coronary arteries according to an example embodiment.

FIG. 2E is intended to show an inflatable device which blocks entrance of blood to the coronary arteries.

FIG. 2E shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 2E shows a device 242 for preventing blood from entering the coronary arteries 104.

FIG. 2E shows the device 242, for example an inflatable balloon or tube 242 shaped and sized to expand against walls of an ascending aorta at exits to coronary arteries, the inflatable balloon or tube 242 blocking entries to the coronary arteries 104, enabling to block blood flow to the coronary arteries when the inflatable balloon or tube 242 is inflated.

In some embodiments, the inflatable balloon or tube 242 includes a hollow center portion 244 which allows blood to flow through.

In some embodiments, the inflatable balloon or tube 242 includes a hollow center portion 244 which allows medical tool(s) to pass through.

Reference is now made to FIG. 2F, which is a simplified line drawing illustration of a device for preventing back flow of debris according to an example embodiment.

In some embodiments, FIG. 2F is intended to show a device which operates as a one way flow valve.

In some embodiments, FIG. 2F is intended to show a device which operates as a filter.

FIG. 2F shows the aorta 102, a direction 256 of blood flow from a heart into the aorta 102, a device including leaflets 252A 252B for preventing back flow of blood, and a control wire 254 for controlling the leaflets to open (252B) and to close (252A).

FIG. 2F show the leaflets in an open, none-debris-blocking state 252A, and next to that in a closed, debris-blocking state 252B.

In some embodiments, the leaflets 252A 252B are flexible, enabling passage of medical tool(s) (not shown) along the leaflets 252A 252b, and yet, when the leaflets are activated to block back flow 252B, the leaflets 252B can lie against the medical tool(s), blocking a space between the leaflets 252B and the medical tool(s), and block blood from flowing back.

In some embodiments, the leaflets 252A 252B are non-porous, blocking blood from flowing back along the aorta.

In some embodiments, the leaflets 252A 252B are porous, configured as a mesh for blocking debris from flowing back along the aorta, yet enabling blood to flow back. In some embodiments, the size of pores in the mesh is selected to be in a range of 120 to 30 microns.

In some embodiments, the leaflets 252A 252B are opened and/or closed by an outside handle acting upon the control wire.

In some embodiments, especially in embodiments which block blood flow back along the aorta to the coronary arteries, the leaflets are close 252B for a short duration, for example in a range of 20 to 40 seconds.

It is noted that the example devices for blocking debris from entering the coronary arteries and/or for blocking backflow of blood toward the coronary arteries are designed to enable operation in conjunction with additional debris-protection devices.

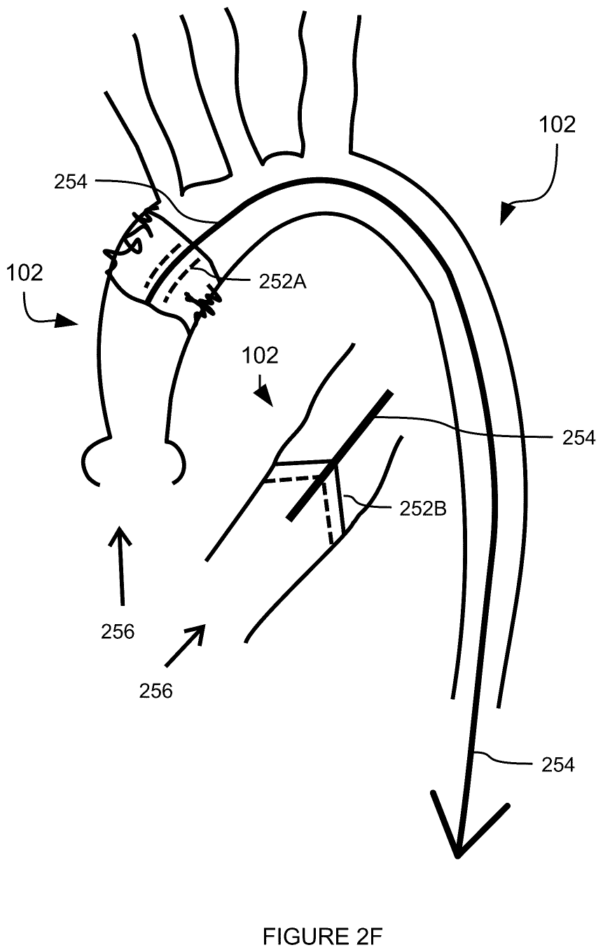

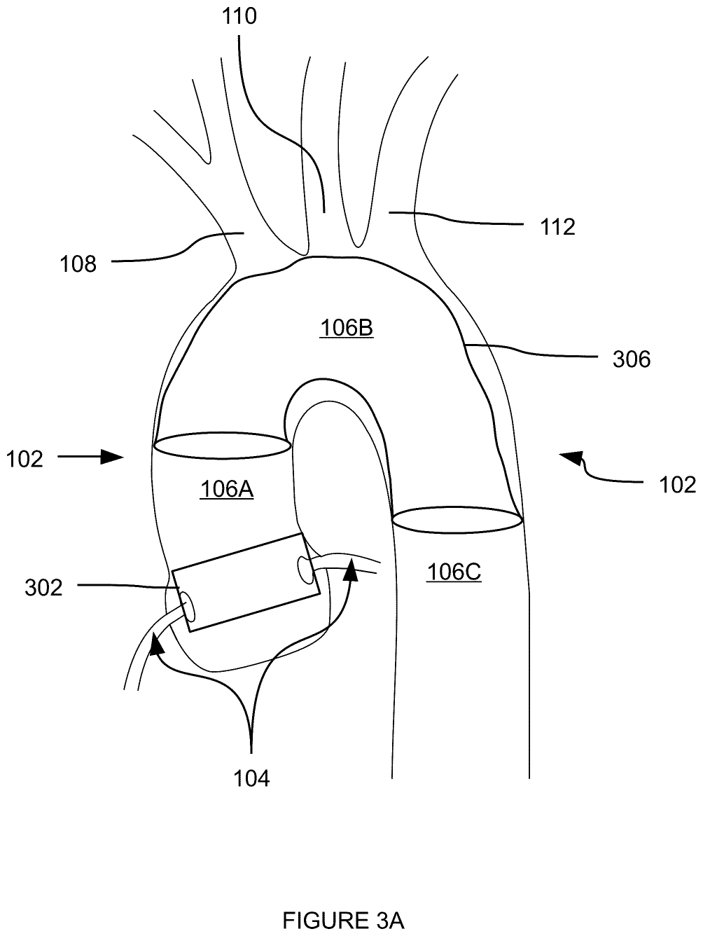

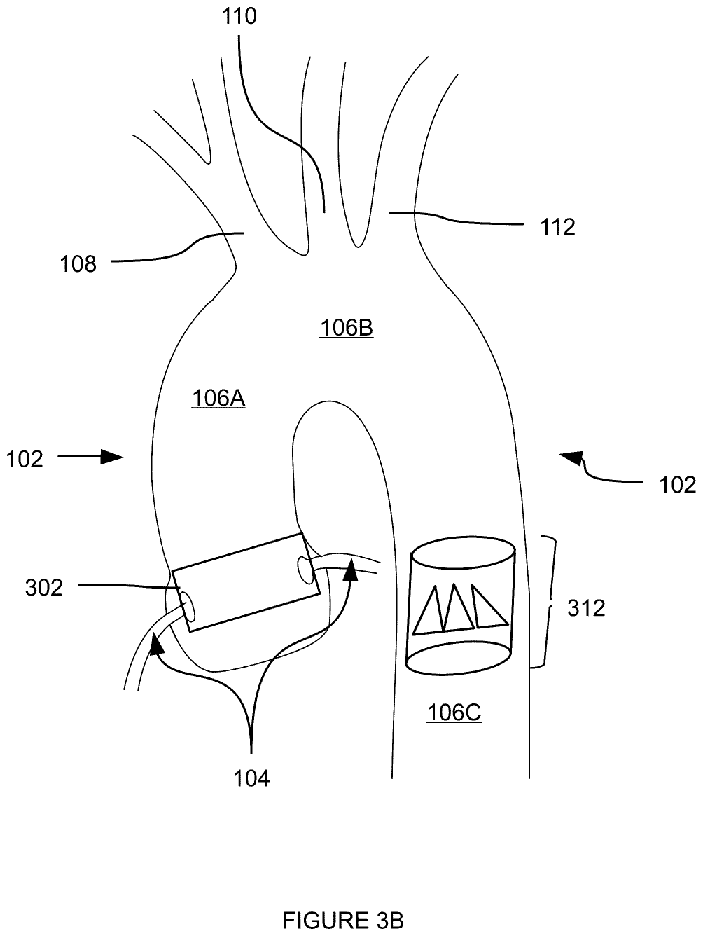

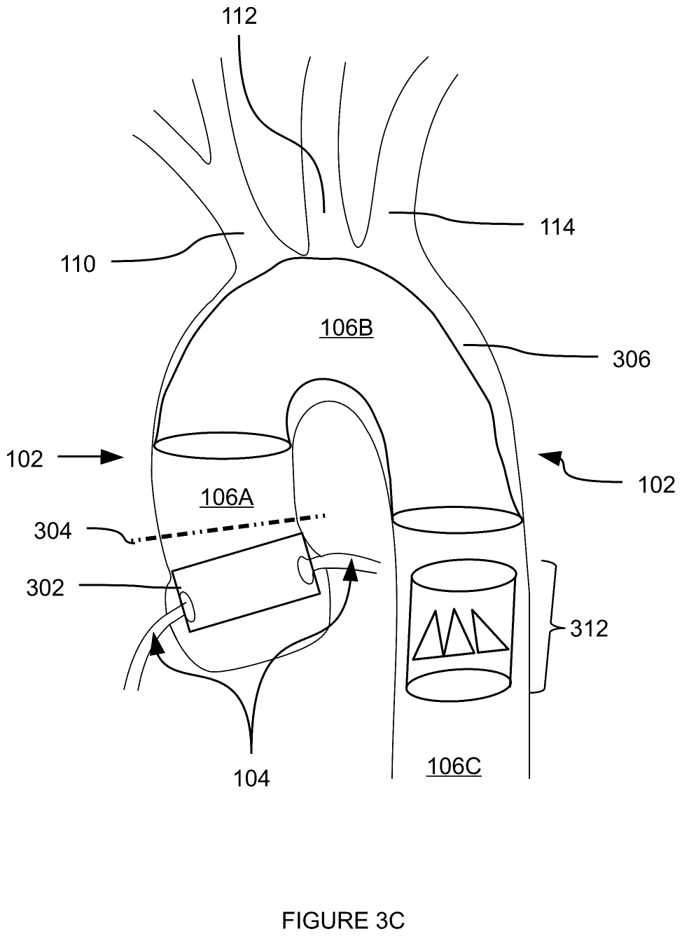

Reference is now made to FIG. 3A, which is a simplified line drawing illustration of a coronary artery protection device located in an aorta in conjunction with a debris-protection device for preventing debris from entering brain arteries according to an example embodiment.

FIG. 3A is intended to show how a coronary artery protection device as described herein can be located and operated in conjunction with, by way of a non-limiting example, an aortic protection device for protecting brain arteries such as described in above-mentioned International Patent Application Publication Number WO 2019/064223 of Brandeis.

FIG. 3A shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 3A shows a coronary artery protection device 302 for preventing blood from entering the coronary arteries 104, and an aortic protection device 306 both located in different portions of the aorta 102.

In some embodiments, the coronary artery protection device 302 may be a device which blocks blood from entering the coronary arteries 104, as shown in FIGS. 2C and 2E.

In some embodiments, the coronary artery protection device 302 may be a device which filters blood entering the coronary arteries 104, as shown in FIG. 2D.

In some embodiments, the coronary artery protection device 302 may be a one way device which blocks blood from flowing back entering the coronary arteries 104, as shown in FIG. 2A.

In some embodiments, the coronary artery protection device 302 may be a one way device which filters back flow blood which may entering the coronary arteries 104, as shown in FIG. 2B.

Reference is now made to FIG. 3B, which is a simplified line drawing illustration of a coronary artery protection device located in an aorta in conjunction with a debris capture device, according to an example embodiment.

FIG. 3B is intended to show how a coronary artery protection device as described herein can be located and operated in conjunction with, by way of a non-limiting example, a debris capture device, by way of a non-limiting example such as described in above-mentioned U.S. Provisional Patent Application No. 63/398,546 of Brandeis.

FIG. 3B shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 3B shows a coronary artery protection device 302, for example as described herein with reference to any one of FIGS. 2A-2E, and a debris capture device 312, for example as described in above-mentioned U.S. Provisional Patent Application No. 63/398,546.

Reference is now made to FIG. 3C, which is a simplified line drawing illustration of a coronary artery protection device located in an aorta in conjunction with a debris capture device and an aortic protection device, according to an example embodiment.

FIG. 3C is intended to show how a coronary artery protection device as described herein can be located and operated in conjunction with, by way of a non-limiting example, a debris capture device, by way of a non-limiting example such as described in above-mentioned U.S. Provisional Patent Application No. 63/398,546 and an aortic protection device for protecting brain arteries such as described in above-mentioned International Patent Application Publication Number WO 2019/064223 of Brandeis.

FIG. 3C shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 3C shows a coronary artery protection device 302, for example as described herein with reference to any one of FIGS. 2A-2E, a debris capture device 312, for example as described in above-mentioned U.S. Provisional Patent Application No. 63/398,546 and an aortic protection device 306 for protecting brain arteries such as described in above-mentioned International Patent Application Publication Number WO 2019/064223 of Brandeis.

Reference is now made to FIG. 3D, which is a simplified line drawing illustration of a coronary artery protection device located in an aorta in conjunction with a debris capture device and an aortic protection device, according to an example embodiment.

FIG. 3D is intended to show how a coronary artery protection device as described herein can be located and operated in conjunction with, by way of a non-limiting example, a debris capture device, by way of a non-limiting example such as described in above-mentioned U.S. Provisional Patent Application No. 63/398,546 and an aortic protection device for protecting brain arteries such as described in above-mentioned International Patent Application Publication Number WO 2019/064223 of Brandeis.

FIG. 3D shows the aorta 102, arteries branching off from the aorta: coronary arteries 104, the brachiocephalic artery 108, the left common carotid artery 110 and the left subclavian artery 112. Portions of the aorta 102 are shown: an ascending aorta 106A, aortic arch 106B and descending aorta 106C.

FIG. 3D shows a coronary artery protection device 302, for example as described herein with reference to any one of FIGS. 2A-2E, a debris capture device 312, for example as described in above-mentioned U.S. Provisional Patent Application No. 63/398,546 and an aortic protection device 306 for protecting brain arteries such as described in above-mentioned International Patent Application Publication Number WO 2019/064223 of Brandeis, where the debris capture device 312 is located inside a lumen formed by the aortic protection device 306.

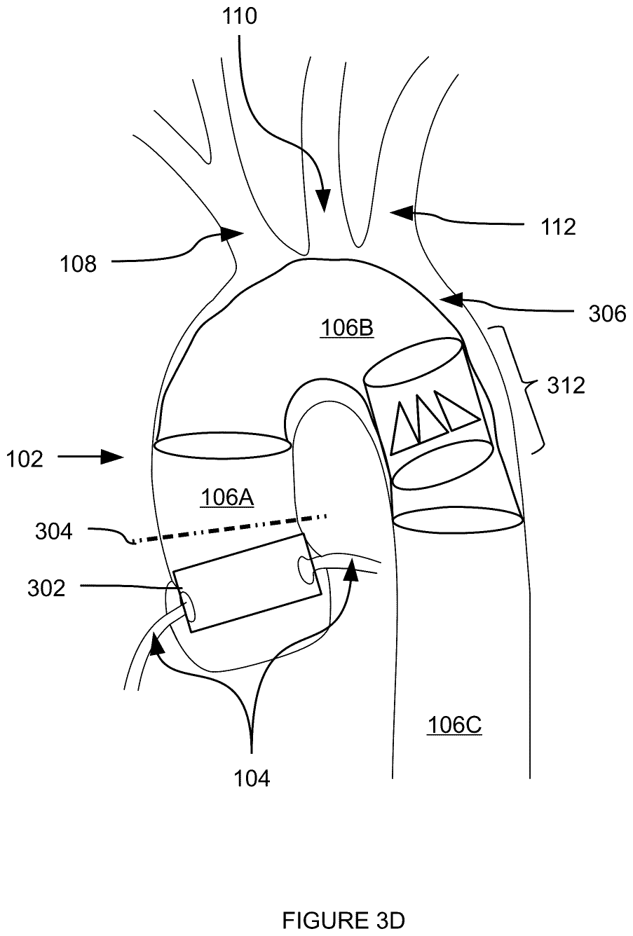

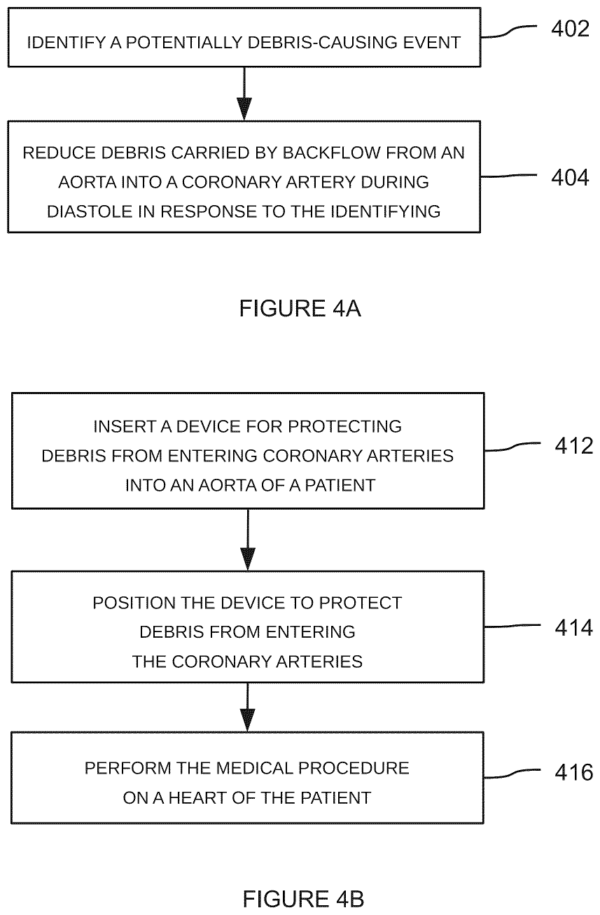

Reference is now made to FIG. 4A, which is a simplified flow chart illustration of a method of reducing damage to the heart during a vascular procedure, according to an example embodiment.

The method of FIG. 4A includes:
identifying a potentially debris-causing event (402); and
reducing debris carried by backflow from an aorta into a coronary artery during diastole in response to the identifying (404).

Reference is now made to FIG. 4B, which is a simplified flow chart illustration of a method for protecting debris from entering coronary arteries during a medical procedure according to an example embodiment.

The method of FIG. 4B includes:
inserting a device for protecting debris from entering coronary arteries into an aorta of a patient (412);
positioning the device to protect debris from entering the coronary arteries (414); and
performing the medical procedure on a heart of the patient (416).

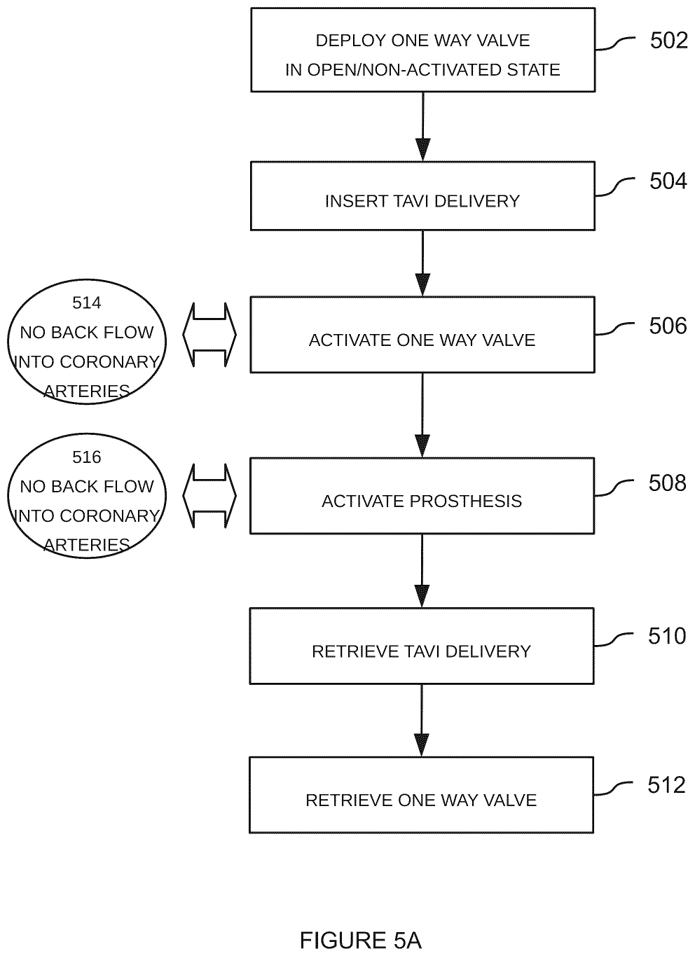

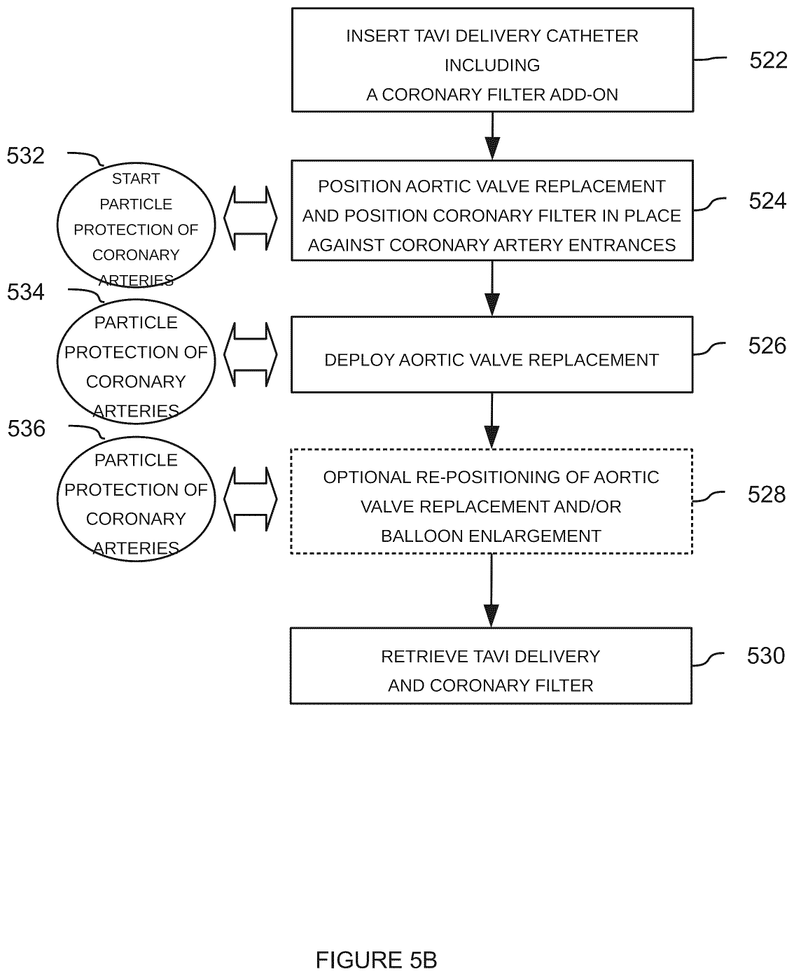

Reference is now made to FIG. 5A, which is a simplified flow chart illustration of a method for preventing back flow of blood into coronary arteries during a medical procedure according to an example embodiment.

The method of FIG. 5A includes:
deploying a one way valve for preventing blood from flowing back into coronary arteries in an aorta of a patient (502);
inserting a TAVI delivery system into the aorta (504);
activating the one way valve to prevent blood from flowing back into the coronary arteries (506);
activating a prosthesis (508);
retrieving the TAVI delivery system from the body of the patient (510); and
retrieving the one way valve from the body of the patient (512).

It is noted that starting from the activating the one way valve (506) there is no back flow of blood to the coronary arteries (514).

It is noted that during the activating the prosthesis (508) there is no back flow of blood to the coronary arteries (516).

In some embodiments, the activating the prosthesis (508) is optionally performed by enlarging a balloon, as is known in the art. The enlarging the balloon is performed under protection, there is no back flow of blood to the coronary arteries (516).

In some embodiments, after activating the one way valve (506) and before activating the prosthesis (508), a physician may need to reposition the prosthesis. The repositioning is optionally performed under protection, there is no back flow of blood to the coronary arteries (516).

When a prosthesis is deployed, in some types of devices—there is a need to fixate the implant to its place—this is done by balloon enlargement. In some cases of blood leakage from sides of the implant-balloon enlargement is used to fix the prosthesis/implant to the heart, stopping the leakage.

During TAVI, a physician may need to correct a position of the prosthesis/implant. For example, an angle of the prosthesis may require adjustment, or the prosthesis may be too deep into the heart or not deep enough or covers entrance to coronaries. In some embodiments, a balloon may be deflated and the implant may be moved forward or backwards, or a “landing” angle may be changed.

Reference is now made to FIG. 5B, which is a simplified flow chart illustration of a method for protecting coronary arteries from entry of particles during a medical procedure according to an example embodiment.

The method of FIG. 5B includes:
insert a TAVI delivery catheter including a coronary filter add on into an aorta of a patient (522);
positioning an aortic valve replacement and positioning a coronary filter in place against coronary artery entrances (524);
deploying the aortic valve replacement (526);
optionally, re-positioning the aortic valve replacement and/or performing balloon enlargement (528); and
retrieving the TAVI delivery system and the coronary filter (530)

It is noted that starting from the positioning (524) particle protection of the coronary arteries begins (532).

It is noted that during the deploying (526) particle protection of the coronary arteries continues (534).

It is noted that if the optional re-positioning and/or balloon enlargement occur, (528), particle protection of the coronary arteries continues (536).

Table 1 below shows an example TAVI procedure and a relation of the TAVI procedure to risk of a debris shower and to a state of a coronary artery protection device according to an example embodiment.

TABLE 1

| | | Risk of | |
|---|---|---|---|
| TAVI | | debris | |
| procedure | TAVI | shower at | |
| step | procedure | coronary | Coronary artery protection |
| number | step name | arteries | device state |
| 1 | Insertion of | Low | Not activated |
| | TAVI delivery | | |
| | system | | |
| 2 | Positioning of | Possible | One-way valve - activated; |
| | the TAVI | | Coronary filter activated upon |
| | delivery | | positioning at coronary artery |
| | system at a | | entrances |
| | deployment | | |
| | location | | |
| 3 | TAVI valve | High | One-way valve - activated; |
| | (prosthesis) | | Coronary filter - activated |
| | deployment | | |
| 4 | Optional re- | High | One-way valve - activated; |
| | positioning | | Coronary filter - activated |
| 5 | Balloon | High | One-way valve - activated; |
| | enlargement of | | Coronary filter - activated |
| | the TAVI valve | | (* in some embodiments the |
| | (prosthesis) | | coronary filter may optionally |
| | | | be pulled back to clear space |
| | | | for the valve prosthesis) |
| 6 | Retrieval of the | Low | In some embodiments, a one |
| | TAVI delivery | | way valve or filter may be |
| | system | | retrieved after retrieval of |
| | | | TAVI delivery system. |
| | | | In some embodiments, a one |
| | | | way valve or filter may be |
| | | | retrieved may be retrieved from |
| | | | together with TAVI delivery |
| | | | system |

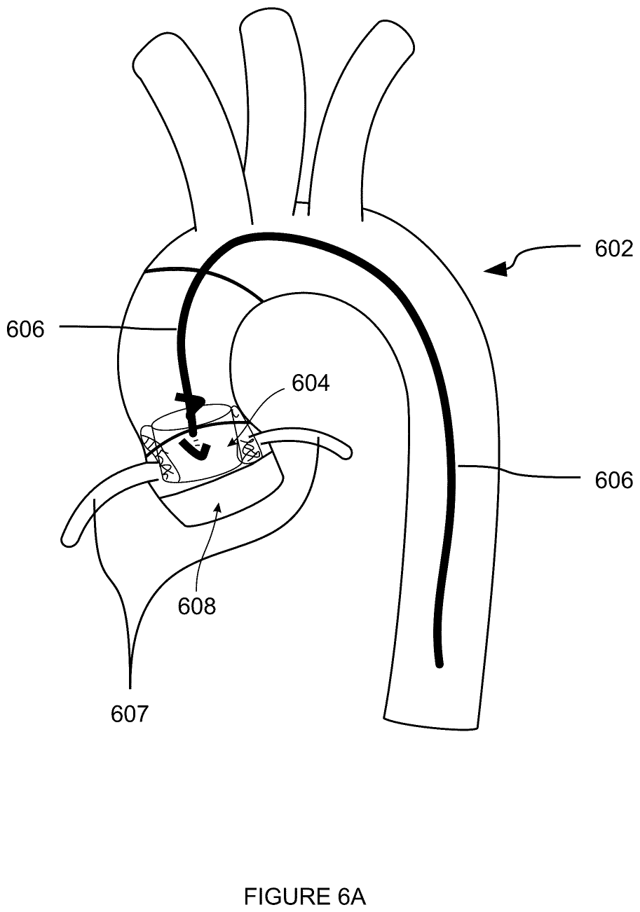

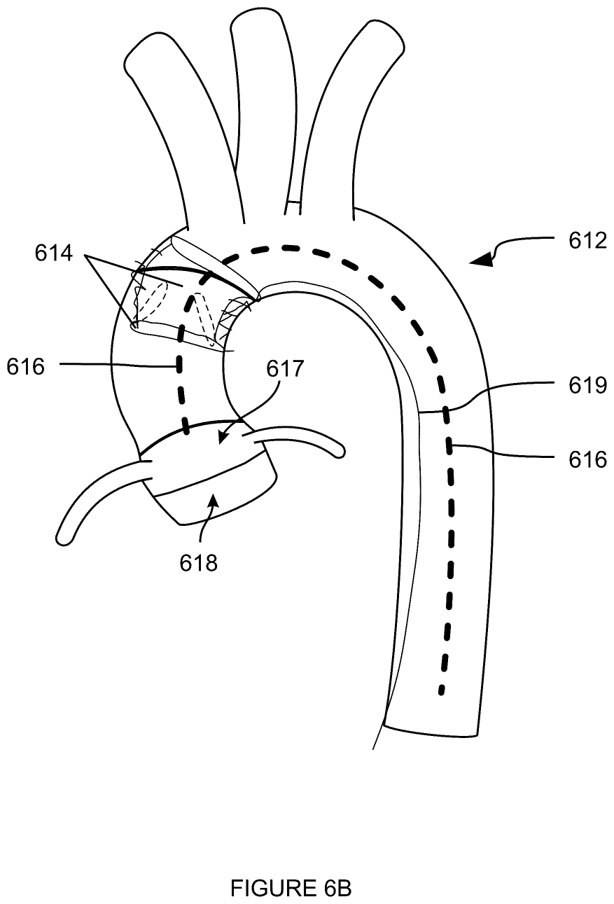

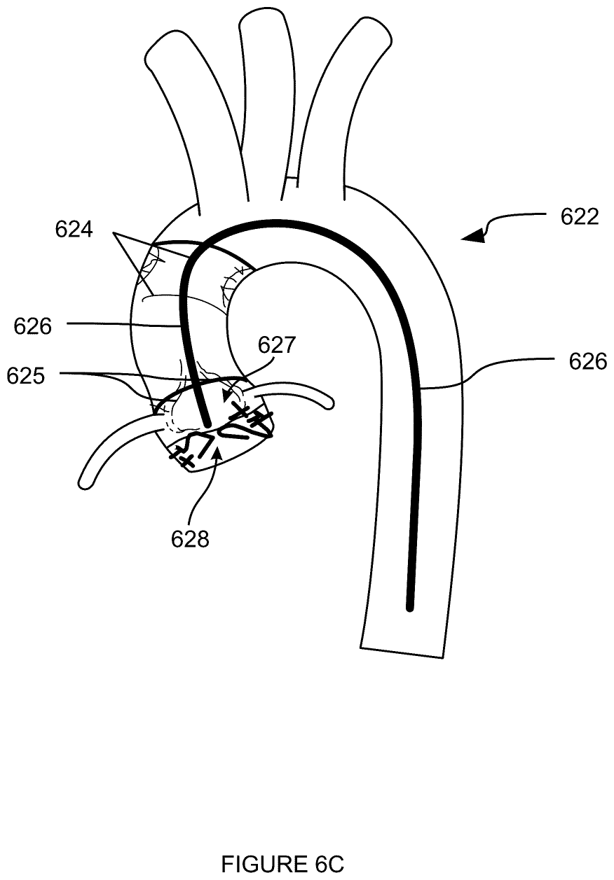

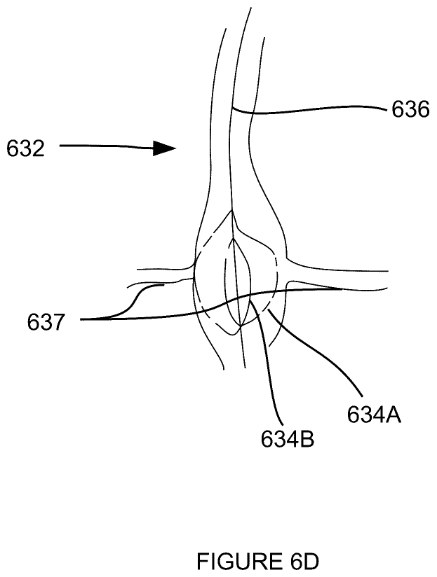

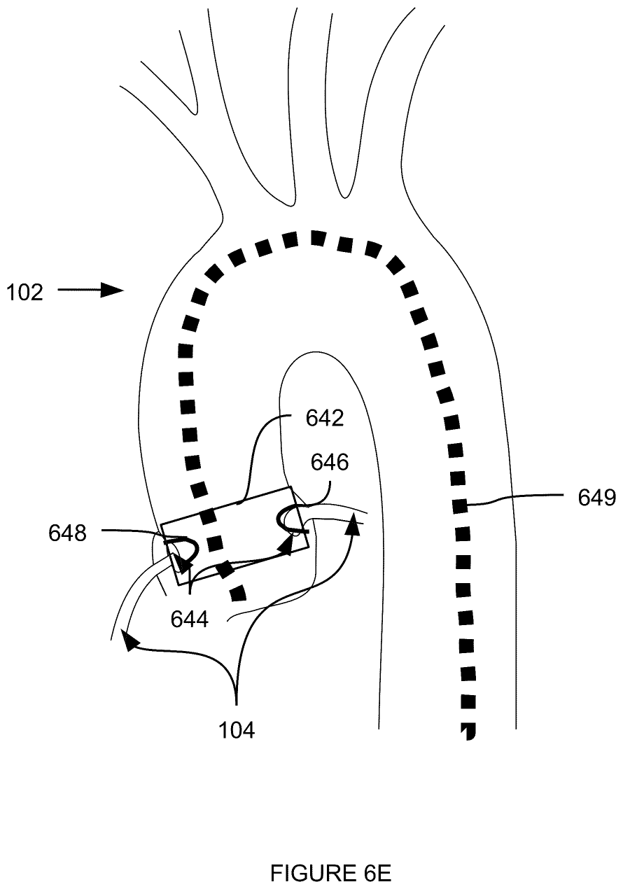

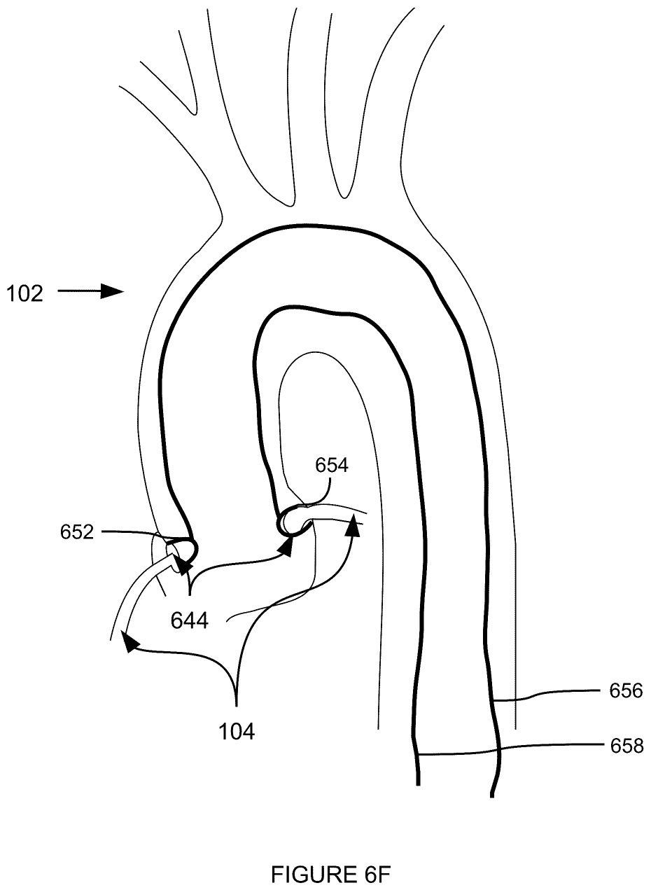

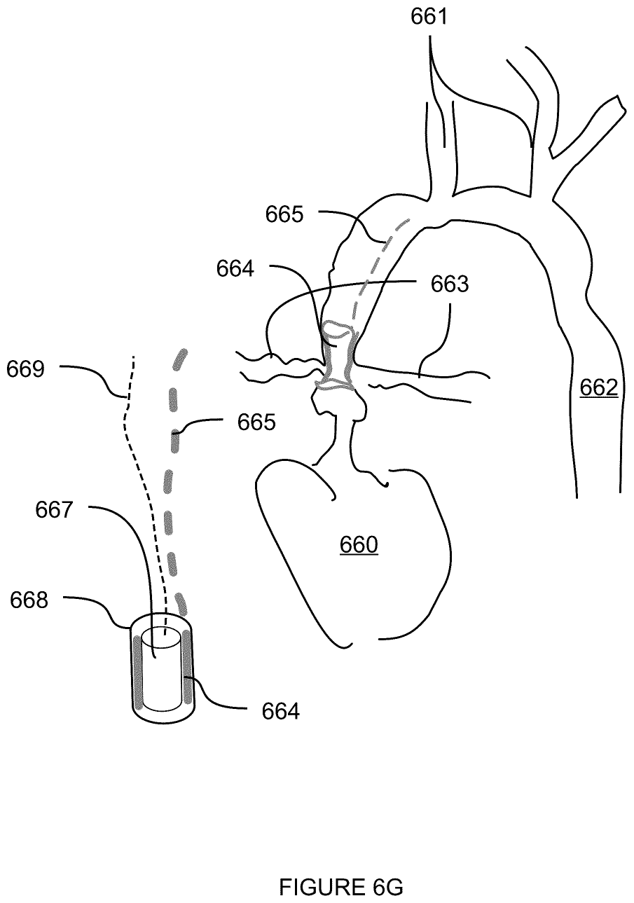

Reference is now made to FIG. 6A, which is a simplified line drawing illustration of a device protecting coronary arteries according to an example embodiment.

FIG. 6A is intended to show a device for protecting coronary arteries which includes a filter filtering blood at entrances to the coronary arteries.

FIG. 6A shows an aorta 602, into which has been inserted a TAVI delivery system 606, placing a cardiac valve prosthesis 608 in situ. FIG. 6A also shows a filter device 604 in place for protecting coronary arteries 607.

In some embodiments, the filter device 604 optionally slides along a same TAVI delivery system 606 as the cardiac valve prosthesis 608.

Reference is now made to FIG. 6B, which is a simplified line drawing illustration of a device protecting coronary arteries according to an example embodiment.

FIG. 6B is intended to show a device for protecting coronary arteries which includes a one way valve.

FIG. 6B shows an aorta 612, into which has been inserted a TAVI delivery system 616, placing a cardiac valve prosthesis 618 in situ. FIG. 6B also shows a one way valve device 614 in place for protecting coronary arteries 617.

In some embodiments, the one way valve device 614 optionally slides along a same TAVI delivery system 616 as the cardiac valve prosthesis 618.

FIG. 6B also shows an optional control wire 619, for controlling activation of the one way valve device 614.

Reference is now made to FIG. 6C, which is a simplified line drawing illustration of devices protecting coronary arteries according to an example embodiment.

FIG. 6C is intended to show devices for protecting coronary arteries which includes a one way valve and a filter for protecting coronary artery entrances.

FIG. 6C shows an aorta 622, into which has been inserted a TAVI delivery system 626, placing a cardiac valve prosthesis 628 in situ. FIG. 6C also shows a one way valve device 624 in place for protecting coronary arteries 627, as well as a filter 625 for protecting coronary artery 627 entrances.

In some embodiments, the one way valve device 624 optionally slides along a same TAVI delivery system 626 as the cardiac valve prosthesis 628.

In some embodiments, the filter 625 for protecting coronary artery 627 entrances optionally slides along a same TAVI delivery system 626 as the cardiac valve prosthesis 628.

Reference is now made to FIG. 6D, which is a simplified line drawing illustration of devices protecting coronary arteries according to an example embodiment.

FIG. 6D is intended to show a device for protecting coronary arteries which includes a double filter for protecting coronary artery entrances.

FIG. 6D shows an aorta 632, into which has been inserted a TAVI delivery system 636. FIG. 6D also shows a filter for protecting coronary artery 637 entrances, the filter including a first, outer mesh layer 634A and a second, inner mesh layer 634B.

In some embodiments, the first, outer mesh layer 634A and the second, inner mesh layer 634B may optionally possess meshes with different pore sizes.

In some embodiments, each one of the first, outer mesh layer 634A and the second, inner mesh layer 634B may optionally be activates separately, that is, one of the layers may be activated to filter and the other layer not activated to filter.

In some embodiments, both of the first, outer mesh layer 634A and the second, inner mesh layer 634B may optionally be activates together, that is, both of the layers may be activated to filter or de-activated from filter.

In some embodiments, the filter device optionally slides along a same TAVI delivery system 636 as the cardiac valve prosthesis (not shown).

Reference is now made to FIG. 6E, which is a simplified line drawing illustration of a device for protecting coronary arteries according to an example embodiment.

FIG. 6E is intended to show a device for protecting the coronary arteries at an ostium, or entrance, to the coronary arteries, which includes one or more filters for protecting the coronary artery ostium, or entrance.

FIG. 6E shows an aorta 102, into which has been inserted a device 642 for protecting coronary artery 104 entrances 644.

The device 642 is sized and shaped to be located to cover the entrances 644 to the coronary arteries 104.

The device 642 includes one or more filters 646 and 648 located in the device 642 to correspond to locations of the entrances 644 to the coronary arteries 104.

In some embodiments, as a non-limiting example, in some cases where protecting the coronary arteries is performed in relation to a TAVI procedure, the device 642 is optionally slid along an outside of a tube 649 used for the TAVI operation. In some embodiments the TAVI tube 649 is optionally brought up to the aortic valve, then the device 642 is optionally slid along the outside of the tube 649, and the device is optionally placed so that the filters 646 648 cover entrances 644 to the coronary arteries 104.

Reference is now made to FIG. 6F, which is a simplified line drawing illustration of a device for protecting coronary arteries according to an example embodiment.

FIG. 6F is intended to show one or more devices for protecting the coronary arteries at an ostium, or entrance, to the coronary arteries, which includes a filter for protecting the coronary artery ostium, or entrance, and a control wire for placing the device at the ostium of the coronary arteries.

FIG. 6F shows an aorta 102, into which have been inserted, by way of a non-limiting example, two devices for protecting coronary artery 104 entrances 644.

Each one of the devices include a mesh 652 654 sized and shaped to cover the entrances 644 to the coronary arteries 104, and a control wire and/or guide wire 656 658 to guide the mesh 652 654 to the ostium of the coronary arteries.

In some embodiments, as a non-limiting example, in some cases where protecting the coronary arteries is performed in relation to a TAVI procedure, the mesh(es) 652 654 are optionally slid along an outside of a tube (not shown, see FIG. 6E) used for the TAVI operation. In some embodiments the TAVI tube is optionally brought up to the aortic valve, then the mesh(es) 652 654 are optionally slid along the outside of the tube, and the mesh(es) 652 654 are optionally placed so that the filters mesh(es) 652 654 cover entrances 644 to the coronary arteries 104.

In some medical procedures relating to the aortic valve, the aortic valve leaflets may be pushed and cover the entrances to one or more of the coronary arteries. The covering of the entrances may be undesirable, especially if the covering lasts for a period of time which may cause damage to the heart, due to lack of blood to blood-vessels of the heart.

The tools described herein for protecting coronary arteries potentially push aortic valve leaflets away from the coronary artery entrances.

In some embodiments, the tools described herein for protecting coronary arteries can potentially be used to push aortic valve leaflets away from the coronary artery entrances.

In some embodiments, filters such as the filters 646 and 648 shown in FIG. 6E and filters 652 and 654 such as shown in FIG. 6F, are optionally designed to form cages jutting away from a wall of the aorta, so that aortic valve leaflets are kept away from the wall of the aorta, and optionally prevented from blocking the entrances to the coronary arteries.

In some embodiments, filters such as the filters 646 and 648 shown in FIG. 6E and filters 652 and 654 such as shown in FIG. 6F, are optionally designed to include a part jutting into the ostium of the coronary arteries, a distance in a range between 0, 5 and up to 20 millimeters.

Jutting into the ostium of the coronary arteries can potentially maintain location of the filters at the entrances to the coronary arteries.

Reference is now made to FIG. 6G, which is a simplified line drawing illustration of a device for protecting coronary arteries according to an example embodiment.

FIG. 6G is intended to show a component and a method for pushing walls of a device for protecting the coronary arteries against artery walls.

FIG. 6G shows a device 664 for protecting the coronary arteries 663, in place at entrances to the coronary arteries 663. FIG. 6E also shows a heart 660, arteries 661 leading to a brain, and the descending aorta 662.

In some embodiments a balloon 667 is inserted into the device 664, and the balloon is expanded in order to push walls of the device 664 against artery walls 668, as shown on a left side of FIG. 6G.

In some embodiments the device 664 optionally includes a control wire 665.

In some embodiments the balloon 667 optionally includes a control wire 669, and/or a tube 669 for inflating and/or deflating the balloon 667.

It is expected that during the life of a patent maturing from this application many relevant medical procedures will be developed and the scope of the term medical procedure is intended to include all such new technologies a priori.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A device for protecting debris from entering coronary arteries, the device comprising:
an expandable frame; and
a debris blocking obstacle,
wherein the expandable frame is shaped and sized to expand against walls of an ascending aorta at a location selected from a group consisting of:
exits to the coronary arteries; and
just downstream of the exits to the coronary arteries; and
wherein the debris blocking obstacle comprises a one way obstacle shaped and sized to prevent particles from being swept back upstream toward coronary arteries of a heart during diastole.

2. (canceled)

3. The device according to claim 1, wherein the debris blocking obstacle comprises a solid section of the frame at the entries to the coronary arteries, enabling to block blood flow to the coronary arteries when the frame is expanded.

4. The device according to claim 1, wherein the debris blocking obstacle comprises an expandable balloon having a hollow tubular shape, the balloon shaped and sized to such that when the balloon is expanded, the balloon presses against aorta walls and blocks blood flow to the coronary arteries; and wherein the balloon comprises flexible protrusions shaped and sized so that when the balloon is not inflated, the protrusions maintain a flow path for blood to flow around the balloon.

5. (canceled)

6. The device according to claim 1, wherein the debris blocking obstacle comprises a mesh with pores sized to prevent particles larger than 30 microns+/−10 microns to pass therethrough, the mesh attached to the frame,
wherein when the frame is expanded against walls of the ascending aorta at exits to coronary arteries, the mesh filters blood entering coronary arteries;
wherein the mesh is designed such that a pore size of the mesh can be controlled to change.

7. (canceled)

8. The device according to claim 6, wherein the mesh is designed to change pore size from a range of 120-150 microns to a range of 30-50 microns and/or designed to completely close.

9-10. (canceled)

11. The device according to claim 1, wherein the one way obstacle comprises one or more of
a. a one way valve;
b. a flap enabling flow downstream and blocking flow upstream; and
c. a mesh which prevent upstream flow of debris;
d. flaps which, when blood attempts to flow back upstream during diastole, the flaps are swept by the blood and extend across the aorta, blocking backward flow of the blood;
e. mesh flaps which, when blood attempts to flow back upstream during diastole, the flaps are swept by the blood and extend across the aorta, blocking backward flow of debris and allowing at least some of the blood to flow back; and
f. both impermeable flaps and mesh flaps, which, when blood attempts to flow back upstream during diastole, the flaps are swept by the blood and extend across the aorta, the impermeable flaps obstructing backward flow of blood, and the mesh flaps allowing at least some blood to flow back; and wherein the impermeable flaps and the mesh flaps are individually controllable.

12-13. (canceled)

14. The device according to claim 1, comprising a control wire for controlling activation and de-activation of the one way obstacle.

15-18. (canceled)

19. The device according to claim 1, wherein the obstacle is designed such that the obstacle can be activated to change from obstructing particles from being swept back upstream during diastole to not obstructing particles from being swept back upstream during systole.

20. The device according to claim 1, wherein the obstacle comprises radio opaque markers to enable detecting whether the obstacle is activated to block or not.

21. The device according to claim 1, wherein the device comprises a combination of more than one type of debris blocking obstacle.

22. The device according to claim 1, wherein the device is configured to be integrated with one or more of:
a. a TAVI/TAVR delivery system;
b. a TAVI/TAVR guide wire;
c. a cerebral embolic protection (CEP) or embolic protection device (EPD).

23-24. (canceled)

25. The device according to claim 1, wherein the expandable frame comprises one or more of shape memory metal and polymer.

26. (canceled)

27. The device according to claim 1, wherein the device is configured to one or more of:
a. enable medical tools to pass along the device and upstream of the device when the device is located in an aorta;
b. enter a patient's body together with medical tools for operating on a heart.

28. (canceled)

29. The device according to claim 1, wherein the expandable frame is configured such that expansion of the frame against walls of the aorta anchors the device to resist movement along a direction of blood flow.

30. A method of reducing damage to the heart during a vascular procedure comprising:
identifying a potentially debris-causing event; and
reducing debris carried by backflow from an aorta into a coronary artery during diastole in response to the identifying.

31. The method according to claim 30, wherein the identifying comprises one or more of:
a. identifying a step of positioning a prosthesis performed in a prosthesis implant procedure;
b. identifying a step of re-positioning a prosthesis performed in a prosthesis implant procedure;
c. identifying a step of pace-up performed in a prosthesis implant procedure;
d. identifying a step of pace-down performed in a prosthesis implant procedure; and
e. identifying a step of inflating a balloon performed in a prosthesis implant procedure.

32-35. (canceled)

36. The method according to claim 30, wherein the reducing debris comprises blocking blood entrance to coronary arteries.

37. The method according to claim 30, wherein the reducing debris comprises filtering blood entering coronary arteries.

38. The method according to claim 30, wherein the reducing debris comprises activating a one-way valve preventing blood from flowing back from the aorta to the coronary artery.

39. The method according to claim 30, wherein the reducing debris comprises activating a one-way filter preventing blood from flowing back from the aorta to the coronary artery.

40-50. (canceled)
